(19) United States
(12) Patent Application Publication
(10) Pub. No.: US 2016/0310942 A1
(43) Pub. Date: Oct. 27, 2016

(54) PRESSURE-BASED CONTROL OF FLUID VOLUMES AND ASSOCIATED DEVICES, SYSTEMS, AND METHODS

(71) Applicant: University of Washington, Seattle, WA (US)

(72) Inventors: Paul Yager, Seattle, WA (US); Joshua Buser, Seattle, WA (US); Samantha Byrnes, Seattle, WA (US); Erin K. Heiniger, Seattle, WA (US); Peter C. Kauffman, Bainbridge Island, WA (US); Paula Ladd, Seattle, WA (US)

(21) Appl. No.: 15/135,461

(22) Filed: Apr. 21, 2016

Related U.S. Application Data
(60) Provisional application No. 62/150,690, filed on Apr. 21, 2015.
(60) Provisional application No. 62/199,777, filed on Jul. 31, 2015.

(51) Int. Cl. B01L 3/00; C12Q 1/68

(52) U.S. Cl. CPC B01L 3/50273; B01L 3/502715; B01L 3/502761; C12Q 1/6844; B01L 3/502738; B01L 2300/0672; B01L 2200/06; B01L 2400/0406; B01L 2300/069

(57) ABSTRACT

The present technology is directed to capillarity-based devices for performing chemical processes and associated system and methods. In one embodiment, for example, a device can include a first porous element having a first pore size and configured to receive a fluid at its proximal portion, and a second porous element having a second pore size greater than the first pore size and configured to receive a fluid at its proximal portion. The first porous element can be positioned across the second porous element such that an overlapping region exists between the porous elements where the porous elements are in fluid communication. Before delivery of the fluid to the second porous element, the fluid pressure at the overlapping region is greater than the capillary pressure of the second porous element such that a fluid delivered to the first porous element wicks through its overlapping portion without wetting the second porous element.

### CROSS-REFERENCE TO RELATED APPLICATIONS

This application claims the benefit of the following pending applications:

(a) U.S. Provisional Application No. 62/150,690, filed Apr. 21, 2015; and

(b) U.S. Provisional Application No. 62/199,777, filed Jul. 21, 2015.

All of the foregoing applications are incorporated herein by reference in their entireties.

The technology in the present application is also related to the subject matter of the following applications, which are also incorporated herein by reference in their entireties:

(a) U.S. Provisional Application No. 61/755,134, filed Jan. 22, 2013;

(b) U.S. Provisional Application No. 61/808,106, filed Apr. 3, 2013;

(c) U.S. Provisional Application No. 61/832,356, filed Jun. 7, 2013;

(d) U.S. Provisional Application No. 61/861,055, filed Aug. 1, 2013;

(e) U.S. Provisional Application No. 61/867,941, filed Aug. 20, 2013;

(f) U.S. Provisional Application No. 61/867,950, filed Aug. 20, 2013; and

(g) U.S. Provisional Application No. 61/868,006, filed Aug. 20, 2013.

Further, components and features of embodiments disclosed in any of the applications incorporated by reference may be combined with various components and features disclosed and claimed in the present application.

### TECHNICAL FIELD

The present technology is generally related to capillarity-based devices for performing chemical processes and associated systems and methods. In particular, several embodiments are directed toward pressure-based delivery of fluid volumes and associated devices, systems, and methods.

### BACKGROUND

Lateral flow strip tests (“LFT” or “LFTs”) have been identified as a diagnostic technology well-suited for point-of-care (“POC”) use in low resource settings. With fluid transport occurring due to the capillary pressure of the strip material (rather than through the use of pumps), LFTs are entirely disposable, rapid, user-friendly and affordable. Numerous LFTs have been developed and successfully used in limited-resource settings, with applications including pregnancy testing and disease diagnosis. The basic function of a LFT is to mix a substance of interest (e.g., an analyte) with a visible label (e.g., antibodies conjugated to gold nanoparticles) and capture the analyte-label complex at a detection line via an immobilized capture molecule (e.g., antibody). While the simplicity of LFTs makes them ideal for use as a POC tool, it has generally limited them to performing tests that can be carried out in a single chemical step. Moreover, the use of LFTs as a clinically relevant diagnostic tool can be limited to targets with high(er) concentrations because of limited analytical sensitivity of the LFT format.

Porous membranes are often used in conventional LFTs and flow-through cartridges. As such, flow of fluid through the LFT usually occurs by wicking through a membrane (either laterally or transversely) onto an absorbent pad. Immunoassays take advantage of such porous membrane systems to measure and analyze analyte samples. The dependence on wicking to generate flow greatly limits control over assay conditions. Specifically, lateral flow assays are often limited to a single step in which the sample (and buffer) is added to the sample pad, and the sample flows by capillary action (i.e., wicking) along the pad. Capillarity provides the force needed to flow fluid from one point to another, causing reagents stored in dry form to be transported along the device and to pass over regions that contain immobilized capture molecules. These devices are typically restricted to simple one-shot detection chemistries like colored nanoparticles that do not provide the sensitivity possible with multistep-detection chemistries, such as enzymatic amplification. They are also rarely quantitative.

Microfluidic systems that include open fluid channels for the flow of buffers, samples, and reagents can inherently be made much more sophisticated, and it is possible to use them to carry out a very large number of fluid-processing steps. Such microfluidic systems usually incorporate a complex disposable, which leads to unavoidably high per-test manufacturing costs and the need for expensive external pumps and valves to move fluids. While microfluidic devices can inherently be very flexible in the functions that they perform, they are also inherently complicated and expensive. Additionally, the devices that have been made that support complex function are usually quite complex themselves. For example, some polymeric laminate cartridges currently developed contain as many as 23 different layers, each of which must be separately manufactured and bonded to the others.

During the last decade, multiple groups have demonstrated mechanisms for controlling fluid flow in porous networks through a variety of valving options. Materials can be embedded into a membrane to slow or delay flow. Noh et al. utilized varying concentrations of patterned wax to control fluidic timing in porous devices. Lutz et al. developed a different approach by embedding sugar barriers into porous membranes. Higher concentrations of sugars resulted in longer delays for fluid delivery. The Yager, Lutz, and Fu groups have also designed methods for the sequential timed delivery of reagents through two-dimensional paper networks that rely on volume metering. Chen et al. developed a fluidic diode using a combination of hydrophobic and hydrophilic coatings to control direction and sequencing of fluid flow.

Although many of the aforementioned valving systems effectively control fluid flow, they also share some limitations. For example, most of these systems are only able to turn flow on or off, but cannot do both. Many of these systems also introduce an additional reagent (wax, sugar, etc.) into the primary fluid path. These reagents may interfere or completely inhibit sensitive downstream reactions such as nucleic acid amplification.

With these concerns in mind, Toley et al. developed valves that utilize fluid to actuate expanding elements. These elements are outside of the main fluid pathway and do not introduce additional reagents. Also, these expanding valves can turn flow on or off and cause fluid diversion/redirection. This system was able to achieve fluidic delays ranging from 5 seconds to 25 minutes with coefficients of variation of less than 9%. Although effective, these systems are limited to use with a maximum volume of a few hundreds of μLs of input sample, which often times is not enough volume of the sample for capturing and/or purifying the targeted analyte or molecule of interest from the sample (such as urine or blood). Within a urine sample, for example, the first 5-10 mL contains the highest concentration of pathogen biomarkers, as shown by Chernesky et al.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIGS. 1A-1D are a series of time-lapsed front views of a capillary-based fluidic system.

FIG. 2 is an electrical circuit model corresponding to the fluidic system shown in FIGS. 1A-1D.

FIG. 3 is a graph showing migration of the fluid front shown in FIGS. 1A-1D as a function of time for a finite fluid source.

FIG. 4 is a top view of four fluidic devices, each including different source materials.

FIG. 5 is a graph showing the change in wetted length over time for each fluidic system shown in FIG. 4.

FIG. 6 is an electrical circuit model corresponding to the fluidic systems shown in FIG. 4.

FIGS. 7A-7B are scanning electron microscope (“SEM”) images showing the relative pore size of glass fiber and nitrocellulose, respectively, both of which are configured for use with one or more embodiments of the present technology.

FIG. 8 is a graphical illustration of pressure versus position for a porous element when wicking fluid from a well.

FIG. 9A is a top view of a microfluidic device configured in accordance with the present technology.

FIG. 9B shows isometric, isolated views of the first and second porous elements of the microfluidic device of FIG. 9A, with the overlapping portions of each identified by the portions bounded by dotted lines.

FIG. 9C is a graph showing the water retention curves for the first and second porous elements of the microfluidic device shown in FIG. 9A.s

FIGS. 10A-10D illustrate a method of using the microfluidic device shown in FIG. 9A in accordance with the present technology.

FIG. 11A is a graph showing a water retention curve for a nitrocellulose membrane.

FIG. 11B is a plot showing modelled saturation levels during wetting of a nitrocellulose membrane.

FIG. 11C is a plot illustrating the modelled fluid pressure at an overlapping region of intersecting, paper-based porous elements after 60 seconds of fluid delivery.

FIG. 11D shows a graph plotting position (along the y-axis) versus pressure for the porous element configuration shown in FIG. 11C.

FIG. 11E is a graph showing a water retention curve for the glass fiber membrane shown in FIG. 11C.

FIG. 12A is a top view of a microfluidic device configured in accordance with another embodiment of the present technology.

FIG. 12B is a cross-sectional view of a portion of the microfluidic device shown in FIG. 12A, taken along line 12B-12B.

FIGS. 13A-13F illustrate a method for using the microfluidic device shown in FIGS. 12A-12B in accordance with the present technology.

FIG. 14A is a top view of a microfluidic device configured in accordance with yet another embodiment of the present technology.

FIG. 14B is a cross-sectional side view of a portion of the microfluidic device shown in FIG. 14A, taken along line 14B-14B.

FIGS. 15A-15C illustrate a method for using the microfluidic device shown in FIGS. 14A-14B in accordance with the present technology.

FIG. 16 is a partial-schematic top view of a microfluidic device configured in accordance with still another embodiment of the present technology.

FIGS. 17A-17C illustrate a method for using a microfluidic device having an actuator configured in accordance with another embodiment of the present technology.

FIG. 18 is a top view of a microfluidic device configured for automated, serial dilutions configured in accordance with the present technology.

FIG. 19 shows the microfluidic device of FIG. 18 after a first, second, and third fluid have been delivered.

FIG. 20 is a top view of a another embodiment of a microfluidic device configured for automated, serial dilutions configured in accordance with the present technology.

FIGS. 21A-21C illustrate a method for using the microfluidic device shown in FIG. 20 in accordance with the present technology.

### DETAILED DESCRIPTION

The present technology describes various embodiments of devices, systems, and methods for processing, analyzing, detecting, measuring, and separating fluids. The devices can be used to perform these processes on a microfluidic scale, and with control over fluid and reagent transport. In one embodiment, for example, a microfluidic device configured in accordance with the present technology includes a first porous element having a first pore size and configured to receive a first fluid at its proximal portion, and a second porous element having a second pore size greater than the first pore size and configured to receive a second fluid at its proximal portion. The first porous element can be positioned across the second porous element such that an overlapping region exists between the first and second porous elements where the first and second porous elements are in fluid communication. Before delivery of the second fluid to the second porous element, the negative fluid pressure at the overlapping region has a greater magnitude than the capillary pressure of the second porous element. As a result, delivery of a first fluid to the proximal portion of the first porous element causes the first fluid to wick distally through the overlapping region without wetting the overlapping portion of the second porous element. Once a distal portion of the first porous element is sufficiently saturated (as described in greater detail below), a second fluid can be added to the proximal portion of the second porous element. The addition of the second fluid changes the pressure differential at the overlapping region, thereby causing the second fluid to pull the first fluid from the first porous element into the distal portion of the second porous element as the second fluid moves through the overlapping region. Such selective fluid control allows the microfluidic devices of the present technology to process large volumes of fluid (e.g., biological samples) in low-cost, point-of-care applications, such as, for example, the processing of urine and extraction/purification of DNA for the diagnosis of chlamydia and gonorrhea.

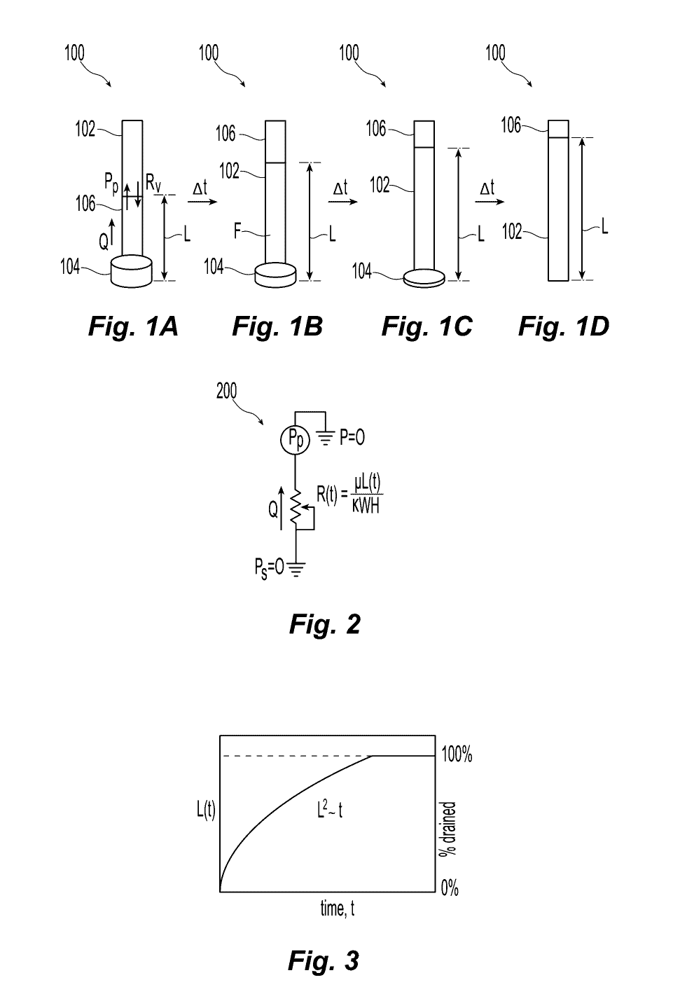

Specific details of several embodiments of the technology are described below with reference to FIGS. 1A-21C. Other details describing well-known structures and systems often associated with capillarity-based devices, biomedical diagnostics, immunoassays, etc. have not been set forth in the following disclosure to avoid unnecessarily obscuring the description of the various embodiments of the technology. Many of the details, dimensions, angles, and other features shown in the figures are merely illustrative of particular embodiments of the technology. Accordingly, other embodiments can have other details, dimensions, angles, and features without departing from the spirit or scope of the present technology. A person of ordinary skill in the art, therefore, will accordingly understand that the technology may have other embodiments with additional elements, or the technology may have other embodiments without several of the features shown and described below with reference to FIGS. 1A-21C.

### I. Definitions

As used herein, “porous element” or “porous membrane” refers to a porous membrane (e.g., a wick, pathway, leg, pad, delivery channel, etc.) through which fluid can travel by capillary action, such as paper, nitrocellulose, nylon, glass fiber, and the like. Unless the context clearly requires otherwise, a porous element can be two-dimensional or three-dimensional (when considering its height in addition to its length and width). Additionally, a porous membrane can be a single layer or may comprise two or more membranous layers. Although in some embodiments a specific term may be used (e.g., “wick,” “pathway,” “leg,” “pad,” “delivery channel,” etc.), it should be understood that use of a different porous element is also within the scope of the present technology.

As used herein, “wettably distinct” means being capable of being wetted by contact with separate fluids without mixing of the fluids at the point of initial wetting. For example, two input legs are wettably distinct if they are physically separated so that each leg could be brought into contact with a separate fluid reservoir. Pathways can be made wettably distinct by a variety of means including, but not limited to, separation via distinct edges (e.g., cut as separate pathways) and separation via an impermeable barrier.

As used herein, “ideal fluid source” or “substantially ideal fluid source” refers to a fluid source that exerts negligible capillary backpressure during release into a porous matrix. One such example of an ideal fluid source is a well source. “Non-ideal fluid source” refers to a fluid source that exerts non-negligible capillary backpressure during release into a porous matrix.

As used herein, a two-dimensional paper network (“2DPN”) refers to a system that includes at least two interconnected wettably distinct wicks, pathways, and/or legs. A one-dimensional paper network (“1DPN”) refers to a system that only includes a single wick, pathway, or leg. A “pseudo-1DPN” refers to a single wick, pathway or leg directly coupled to one or more fluid sources (e.g., without a wettably distinct leg therebetween).

### II. Physical Principles

a. Relationship Between Capillary-Driven Flow and Electrical Circuits

FIGS. 1A-1D are time lapsed front views showing a fluidic system 100 that includes a wick 102 fluidly connected at one end to an ideal fluid source 104. The ideal fluid source 104 contains a finite volume of a fluid F. As shown in FIGS. 1A-1D, fluid F flows from the ideal fluid source 104 to the wick 102 via capillary action. The rate Q at which the fluid F flows through the wick 102 is affected by two opposing forces: (1) the capillary pressure P_{c} of the wick 102 that pulls the fluid F into the wick 102, and (2) the viscous resistance R_{V} that opposes fluid flow through the pores of the wick 102. Viscous resistance R_{V} depends on the wetted length L of the fluid column 106, and is determined by the equation R_{V}=μL/κWH (where μ is fluid dynamic viscosity, κ is permeability, and W is the width of the wick 102 and H is the height of the wick 102). As more fluid F is taken up by the wick 102, the length L of the fluid column 106 increases such that the length L of the fluid column 106 is a function of time, t. Since the viscous resistance R_{V} depends on the wetted length, L(t), viscous resistance R_{V} thus also depends on time (i.e., R_{V}=μL(t)/κWH).

The capillary-driven flow shown in FIGS. 1A-1D can also be described by analogy to simple electrical circuits. For example, as shown in the electrical circuit model 200 in FIG. 2 (Dharmaraja et al.), the pressure P_{c} created by capillary force or gravity can be analogized to electrical voltage, the fluid flow rate Q can be analogized to electrical current, and viscous resistance R_{V} can be analogized to electrical resistance. Atmospheric pressure (i.e., P=0) acts on all fluid-air interfaces and can be analogized to the electrical ground. The capillary backpressure P_{S} exerted by the ideal fluid source 104 can also be represented as a ground since, for ideal fluid sources, the capillary backpressure is negligible and thus essentially zero. Furthermore, just as Ohm's Law (i.e., I=V/R) in electrical circuits relates the current I to the voltage V and resistance R, the one-dimensional form of Darcy's law (i.e., Q=P/R) similarly relates the fluid flow rate Q to the driving pressure P and viscous resistance R. The following equations can be derived from evaluating the circuit model 200:

$$\begin{matrix}Q ( t ) = ɛ WH \frac{d L ( t )}{d t} = \frac{{P}_{P}}{R} = \frac{κ {WHP}_{P}}{μ L ( t )} ; & ( Equation 1 ) \\ {L}^{2} = ( \frac{2 κ {P}_{P}}{μɛ} ) t ; & ( Equation 2 )\end{matrix}$$

where ε is the void volume of the porous material.

The graph shown in FIG. 3 shows the change in wetted length over time for the fluidic system 100. The combination of a constant capillary pressure P_{c} and the rising resistance R_{V} during wet out causes the fluid front to slow over time (following L²∝t) until the fluid source 104 is depleted. The scaling found in Equation 2 (L²∝t) matches exactly the Lucas-Washburn expression describing one-dimensional driven wet-out. Accordingly, electrical analogies can be useful for understanding the basic concepts of capillary-driven flow in porous materials.

b. Non-ideal Fluid Sources and Capillary Backpressure

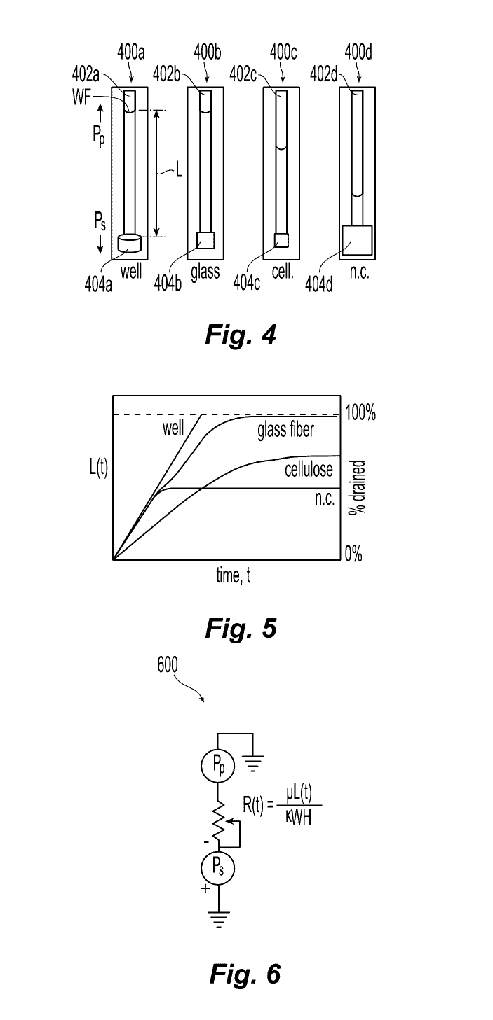

The derivations based on FIGS. 1A-1D assume fluid sources comprised of ideal fluid source materials that exert negligible backpressure during the release of fluid from the source. As illustrated in FIGS. 4-6, although non-ideal fluid source materials can also be utilized, the choice of non-ideal source material can affect both the total amount of fluid released into the wick and the rate at which fluid travels through the wick.

FIG. 4 is a top view of four fluidic systems 400a-d individually comprising different fluid sources 404a-d, each fluidly connected to a nitrocellulose wick 402a-d. FIG. 4 illustrates each fluidic system 400a-d once wicking has ceased in each system. Additionally, each fluid source 404a-d initially held the same volume of fluid. Fluidic system 400a, for example, includes a well 404a (ideal fluid source), fluidic system 400b includes a glass fiber fluid source 404b (non-ideal), fluidic system 400c includes a cellulose fluid source 404c (non-ideal), and fluidic system 400d includes a nitrocellulose fluid source 404d (non-ideal). As shown in FIG. 4, in the fluidic system 400a utilizing the well source 404a (ideal), the entire volume of fluid was released into the wick 402a and extends along a length L of the wick 402a between the input end of the wick 402a (not visible) and its wetting front WF. Out of the non-ideal fluid sources, the glass fiber source 404b also released the entire volume of fluid, yet both the cellulose fluid source 404c and nitrocellulose fluid source 404d retained a large percentage of the fluid.

The graph in FIG. 5 shows that while the glass fiber fluid source 404b fully drained, the release rate was slower than that in the fluidic system utilizing the well source 404a (ideal fluid source). When the nitrocellulose fluid source 404d was used, well-like (ideal) delivery occurred until the nitrocellulose fluid source became about 50% depleted, at which point delivery quickly halted. Furthermore, although the cellulose fluid source 404c delivered a larger percentage of fluid than the nitrocellulose fluid source 404d, the cellulose fluid source 404c showed a much slower release. Accordingly, the non-ideal fluid sources 404b-d exhibited non-linear release profiles (as shown in FIG. 5) and indicate that capillary backpressures can change as fluid is drained.

FIG. 6 is an electrical circuit model 600 (Dharmaraja et al.) that describes the non-ideal fluid source behavior shown in FIGS. 4 and 5. As shown in FIG. 6, when a non-ideal fluid source is used as the fluid source, the fluid source is represented in the circuit model 600 by a voltage source rather than a ground. This is because, in contrast to ideal fluid sources, non-ideal fluid sources exhibit non-negligible capillary backpressure P_{S} (e.g., a pressure that opposes the capillary pressure of the wick).

Using the circuit models for ideal and non-ideal sources described above with reference to FIGS. 2 and 6, the time-dependent capillary pressure of a given material can be derived based on experimentally obtainable values. For example, for a well or ideal fluid source:

$${Q}^{ideal} = ɛ WH \frac{d {L}^{ideal}}{d t} = \frac{{P}_{P}}{R} = \frac{κ {WHP}_{P}}{μ {L}^{ideal}} \qquad ( Equation 3 )$$

For a non-ideal fluid source:

$$\begin{matrix}{Q}^{non \text{-} ideal} = ɛ WHL \frac{d {L}^{non \text{-} ideal}}{d t} \\ = \frac{{P}_{P} - {P}_{S} ( t )}{R} \\ = \frac{κ WH [ {P}_{P} - {P}_{S} ( t ) ]}{μ {L}^{non \text{-} ideal}}\end{matrix} \qquad ( Equation 4 )$$

Taking the ratio of these two equations gives the time dependent capillary pressure of a non-ideal fluid source (P_{S}(t)) based on the length L and rate dL(t)/dt that fluid has traveled through the wick for both the ideal and non-ideal case:

$$\frac{{P}_{S} ( t )}{{P}_{P}} = 1 - \frac{{L ( t )}^{pad} \frac{d {L ( t )}^{pad}}{d t}}{{L ( t )}^{well} \frac{d {L ( t )}^{well}}{d t}} \qquad ( Equation 5 )$$

Accordingly, the capillary pressures of the wick material and fluid source material affect both the distance and rate at which fluid travels through the wick (L(t) and dL(t)/dt, respectively).

c. Effect of Pore Size on Capillary Pressure

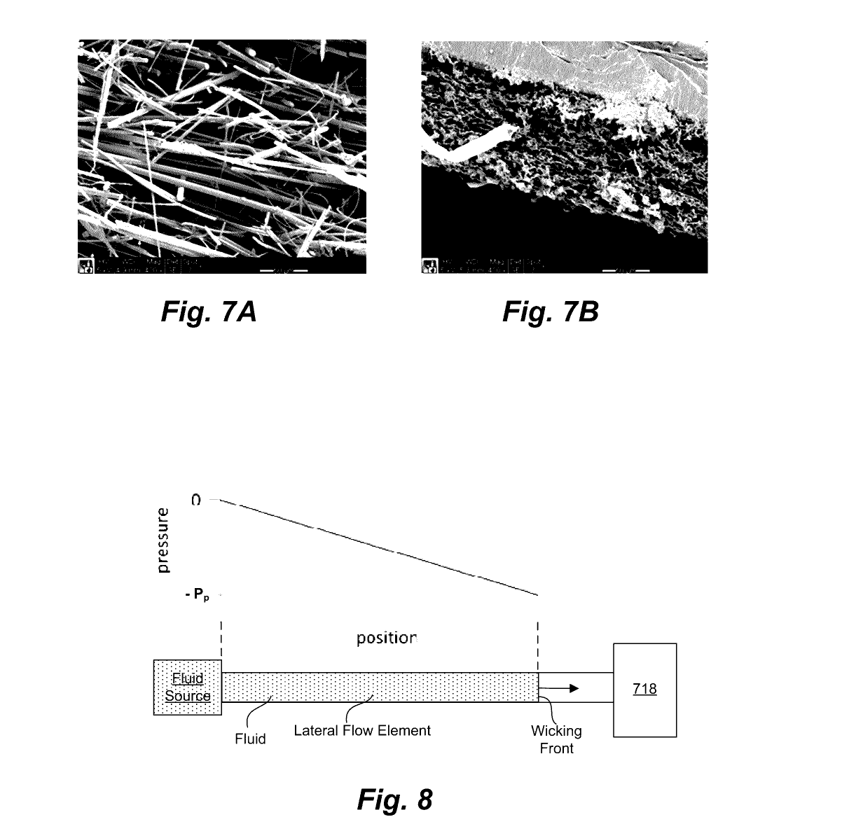

For lateral flow through a wick, capillary pressure P_{c} is governed by the following equation:

$${P}_{c} = \frac{2 γcosθ}{{r}_{m}} ; \qquad ( Equation 6 )$$

where γ is surface tension, θ is the liquid/solid contact angle, and rₘ is the average pore radius of the wick material. As demonstrated by Equation 6, capillary pressure Pₚ is inversely proportional to pore radius rₘ, which varies greatly depending on the wick material. FIGS. 7A and 7B, for example, are SEM images showing the pore size of a glass fiber membrane and a nitrocellulose membrane, respectively. As shown in FIGS. 7A and 7B, the glass fiber membrane has a larger pore size, on average, than the nitrocellulose membrane. As such, the observed capillary pressure P_{c} for glass fiber membranes (about 0.3 kPa) is typically much lower than the observed capillary pressure P_{c} for nitrocellulose (about 3 kPa to about 10 kPa).

FIG. 8 is a schematic showing a porous element as it wicks fluid from a fluid source, and a graph illustrating the corresponding pressure along the length of the wetted portion of the porous element. As shown in FIG. 8, the capillary pressure P_{c} at the fluid source is set to zero, and the pressure decreases linearly until reaching a negative capillary pressure P_{c} at the wetting front. This negative capillary pressure—P_{c} causes the wetting front to advance further into the porous media, as evidenced by Darcy's Law:

$$Q = \frac{κ A Δ P}{μ L} ; \qquad ( Equation 7 )$$

where Q is flow rate, κ is membrane permeability, A is cross sectional area perpendicular to flow, ΔP is the pressure difference between inlet and outlet, μ is the viscosity of the flowing fluid, and L is the length of wetted membrane. However, as discussed in Sections II(a) and II(b) above, the fluid source can exert an opposing capillary backpressure P_{S} which can slow the inhibition of fluids into the porous media. Also, it will be appreciated that although FIG. 8 shows a linear relationship between pressure and position along the porous element, in some embodiments the relationship may be non-linear. For example, porous media having a non-homogeneous pore size and/or non-homogeneous pore distribution will show only a slight decrease in pressure for the majority of the wetted length and then a sharp decrease in pressure at or near the wicking front. As discussed in greater detail below, the microfluidic devices and systems of the present technology utilize the above-described pressure differential to control fluid flow through a paper-based, porous network.

### III. Selected Embodiments of Pressure-Based Control Devices

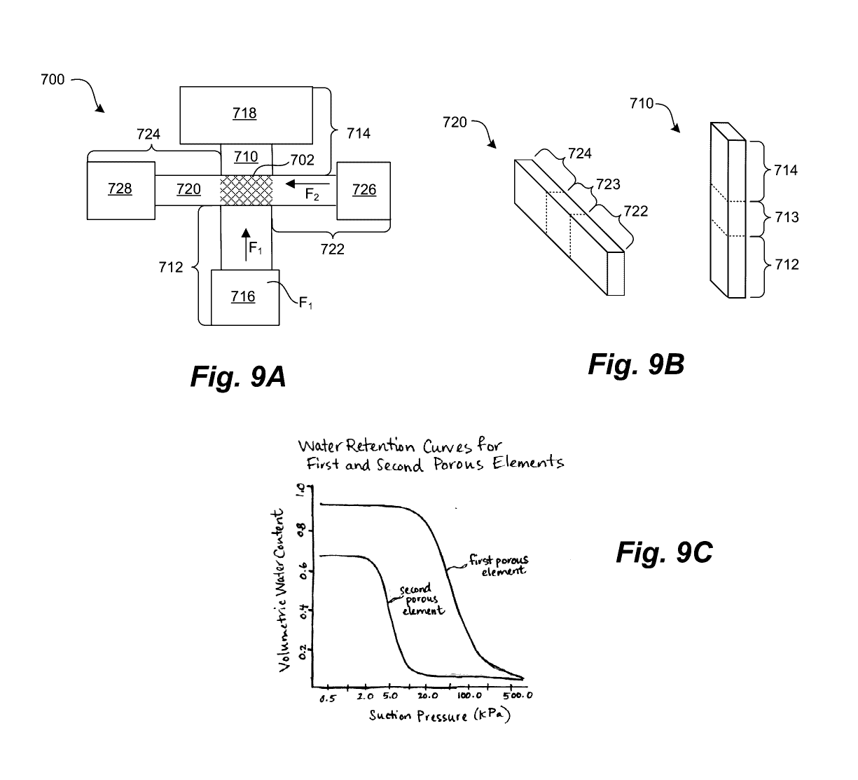

FIG. 9A is a top view of a paper-based microfluidic device 700 (also referred to herein as “device 700”) configured in accordance with an embodiment of the present technology. As shown in FIG. 9A, the device 700 can include a first porous element 710 and a second porous element 720 positioned across the first porous element 710 such that an overlapping region 702 exists between the first and second porous elements 710, 720. FIG. 9B shows isometric views of the first and second porous elements 710, 720 isolated from the device 700 and separated from one another for illustrative purposes. Referring to FIGS. 9A-9B together, the first porous element 710 includes a proximal portion 712 configured to receive one or more fluids, a distal portion 714, and an overlapping portion 713 positioned along the first porous element 710 between the proximal portion 712 and the distal portion 714. The second porous element 720 includes a proximal portion 722 configured to receive one or more fluids, a distal portion 724, and an overlapping portion 723 positioned along the second porous element 720 between the proximal portion 722 and the distal 724.

The proximal portion 712 of the first porous element 710 can include an input portion 716. For example, in the embodiment shown in FIG. 9A, the input portion 716 comprises a separate porous element, such as a source pad (e.g., glass fiber, nitrocellulose, cellulose, etc.) that is positioned in fluid communication with the first porous element 710 and configured to receive a volume of fluid from a fluid source (e.g., a transfer pipette). In other embodiments, however, the proximal portion 712 may not include a separate input portion 716, and the fluid can be delivered directly to the proximal portion 712 of the first porous element 710 and/or one or more fluid sources (such as a well) can be positioned on the first porous element 710.

The distal portion 714 of the first porous element 710 can include a receiving portion 718. For example, in the embodiment shown in FIG. 9A, the receiving portion 718 comprises a waste pad (e.g., cellulose, nitrocellulose, etc.) positioned in fluid communication with the first porous element 710 and configured to receive all or a portion of the delivered volume of fluid after it travels through the first porous element 710. In other embodiments, the distal portion 714 does not include a separate receiving portion 718.

The proximal portion 722 of the second porous element 720 can include an input portion 726. For example, in the embodiment shown in FIG. 9A, the input portion 726 comprises a separate porous element, such as a source pad (e.g., glass fiber, nitrocellulose, cellulose, etc.) that is positioned in fluid communication with the second porous element 720 and configured to receive a volume of fluid from a fluid source (e.g., a transfer pipette). In other embodiments, the proximal portion 722 does not include a separate input portion 726, and the fluid can be delivered directly to the proximal portion 722 of the second porous element 720 and/or one or more fluid sources (such as a well) can be positioned on the second porous element 720.

The distal portion 724 of the second porous element 720 can include a receiving portion 728. For example, in the embodiment shown in FIG. 9A, the receiving portion 728 comprises a waste pad (e.g., cellulose, nitrocellulose, etc.) positioned in fluid communication with the second porous element 720 and configured to receive all or a portion of the delivered volume of fluid after it travels through the second porous element 720. In other embodiments, the distal portion 724 does not include a separate receiving portion 728.

The overlapping portion of the first porous element 710 and the overlapping portion of the second porous element 720 together define the overlapping region 702. For example, the cross-sectional area of the overlapping region 702 is generally equal to the width of the first porous element 710 multiplied by the width of the second porous element 720. In the embodiment shown in FIG. 9A, the second porous element 720 is positioned on top of and in direct contact with the first porous element 710. As such, the overlapping portion of the first porous element 710 is in fluid communication with the overlapping portion of the second porous element 720. In other embodiments, the first porous element 710 can be positioned on top of the second porous element 720. Additionally, in some embodiments the first and second porous elements 710, 720 can be spaced apart, so long as the overlapping portion 713 of the first porous element 710 remains in fluid communication with the overlapping portion 723 of the second porous element 720. For example, the device 700 may include a thin separation material (not shown) positioned between the first and second overlapping portions 713, 723. In some embodiments, the separation material can have one or more openings that are sized and shaped to more precisely control the contact between the first and second porous elements 710, 720.

The first porous element 710 and the second porous element 720 can be selected based on their respective water retention curves (“WRC's”) for a given fluid and medium. As shown in the graph of FIG. 9C, for the same fluid and medium, the second porous element 720 has a WRC that is shifted to the left of the first porous element 710. In other words, for the same fluid, medium, and volumetric water content, the second porous element 720 exhibits a lower suction pressure than the first porous element 710. Each of the porous element's WRC represents a novel, advanced quantitative model that compiles several parameters affecting the negative pressure magnitude at the overlapping region 702, such as contact angle between the delivered fluid and the respective porous element and average pore size of the respective porous element. In the embodiment shown in FIGS. 9A-9B, the first porous element 710 is made of a first porous material having a first average pore size, and the second porous element 720 is made of a second porous material having a second average pore size greater than the first average pore size. In the embodiment shown in FIG. 9A, for example, the first porous element 710 is made of nitrocellulose, and the second porous element 720 is made of glass fiber. In other embodiments, however, the first and second porous elements 710, 720 can be made of other porous materials so long as the WRC of the second porous element 720 is shifted to the left of the WRC of the first porous element 710.

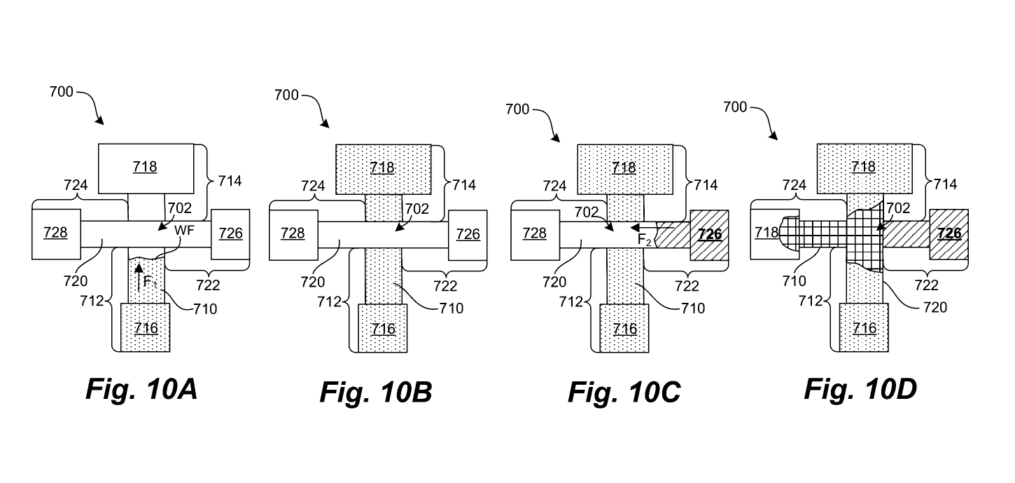

FIGS. 10A-10D are time-lapsed views of the microfluidic device 700 shown in FIG. 9A during various stages of fluid delivery. As shown in FIG. 10A, a first fluid F₁ can be added to the input portion 716 of the first porous element 710 (e.g., using a plastic transfer pipette). The first fluid F₁ then begins wicking distally through the first porous element 710, towards the overlapping region 702. Because of the WRC's of the first and second porous elements 710, 720 (FIG. 9C), the negative fluid pressure magnitude within the overlapping region 702 is such that the wicking front WF of the first fluid F₁ passes through the overlapping region 702 without wetting (or without substantially wetting) the overlapping portion of the second porous element 720, as shown in FIG. 10B. As used herein, the phrase “without wetting” refers to substantially no transfer of fluid between the first and second porous elements 710, 720. As such, the first fluid F₁ will continue to imbibe the first porous element 710 only, thereby leaving the second porous element 720 substantially dry.

Once the distal portion 714 and/or receiving portion 718 of the first porous element 710 becomes sufficiently saturated, a second fluid F₂ can be delivered to the input portion 726 of the second porous element 720. As described in greater detail below, the saturation percentage required at the distal portion 714 before delivering the second fluid F₂ depends on the size, shape of the first and second porous elements 710, 720, as well as the materials used. In general, however, “sufficiently saturated” can be approximated as at least 60% of the fluid volume delivered.

Addition of the second fluid F₂ to the second porous element 720 lowers the magnitude of the negative pressure at the overlapping region 702, thereby allowing the second fluid F₂ to imbibe distally along the second porous element 720. As shown in FIG. 10D, as the second fluid F₂ passes through the overlapping region 702, the second fluid F₂ mixes with first fluid F₁ and pulls a volume of the first fluid F₁ distally along the distal portion 724 of the second porous element 720 to the receiving portion 718.

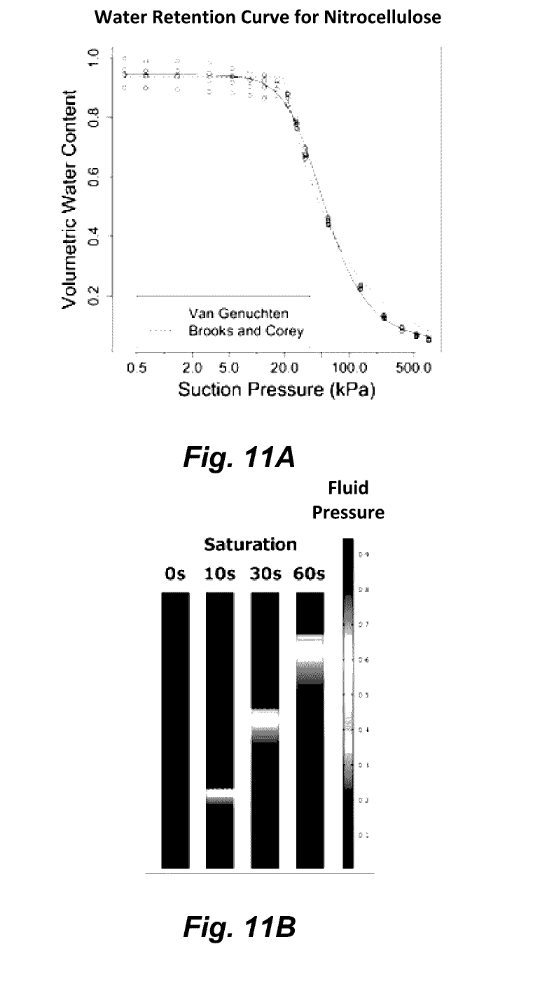

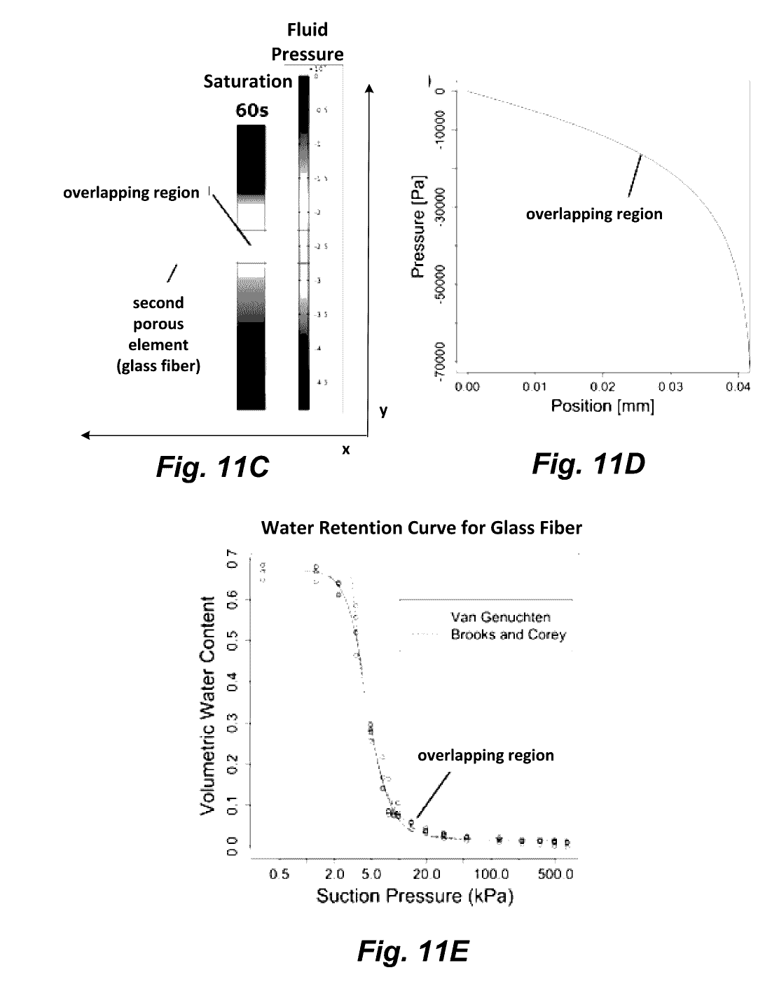

FIGS. 11A-11E are various graphs and charts showing that the pressure differential at an overlapping region between first and second porous elements (arranged as disclosed herein) will cause a fluid delivered to the first porous element to preferentially wick through the first porous element before a second fluid is delivered to the second porous element. FIG. 11A, for example, shows a water retention curve for a nitrocellulose membrane. The measured permeability of the nitrocellulose membrane and the water retention curve data can then be fitted using the Van Genuchten technique. Parameters related to the water retention curve data along with measured membrane permeability can then be used in a Richards equation model implemented through a subsurface flow module, such as COMSOL Multiphysics® Modeling Software. The wetting of the nitrocellulose membrane is shown in FIG. 11B, with the wetting front advancing into the material from a fluid source located at the lower boundary. FIG. 11C shows a chart illustrating the modelled fluid pressure at an overlapping region of intersecting, paper-based porous elements after 60 seconds of fluid delivery. The pressure along the y-axis of the network shown in FIG. 11C is plotted in FIG. 11D. It can be observed that the fluid pressure at the overlapping region is negative (e.g., about 11 kPa in one particular embodiment).

FIG. 11E shows a water retention curve for the glass fiber membrane. As shown in FIG. 11E, at a suction pressure of 11 kPa, very little of the glass fiber membrane is saturated. Accordingly, the glass fiber membrane will not pull significant fluid from the overlapping region while the nitrocellulose membrane is wetting, due to the pressure at the overlapping region.

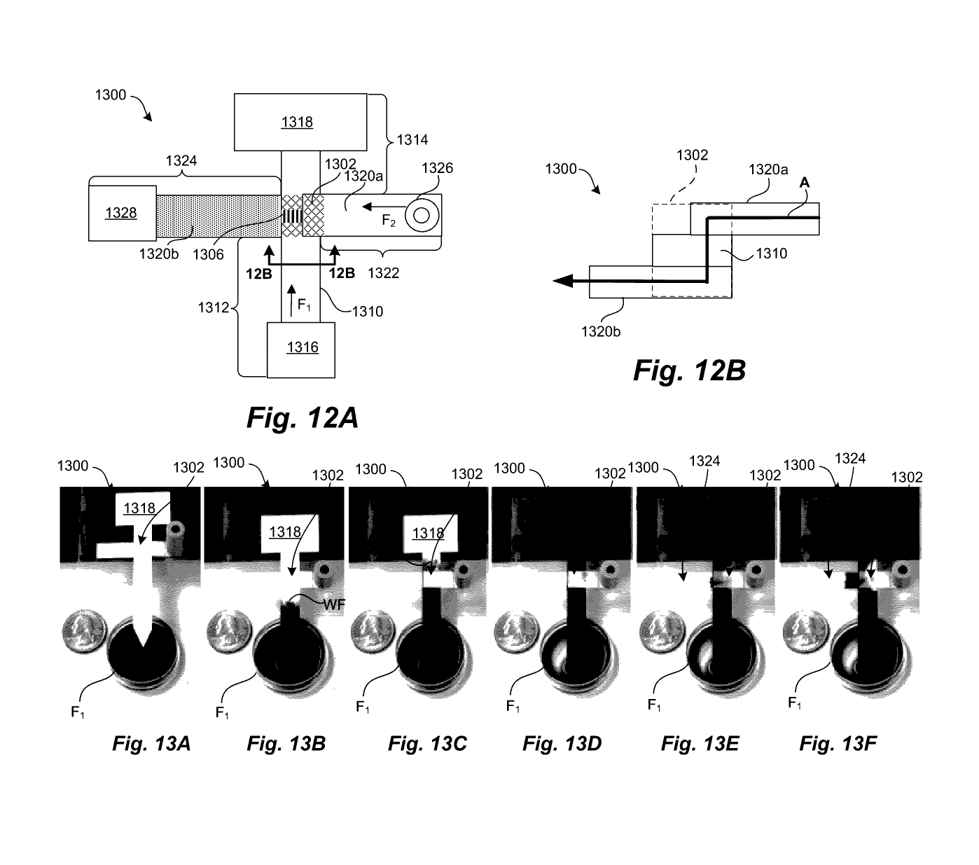

FIG. 12A is a top view of a paper-based microfluidic device 1300 (also referred to herein as “device 1300”) configured in accordance with another embodiment of the present technology. As shown in FIG. 12A, the device 1300 can include a first porous element 1310 and a second porous element 1320 positioned across the first porous element 1310 such that an overlapping region 1302 exists between the first and second porous elements 1310, 1320. The first porous element 1310 includes a proximal portion 1312 configured to receive one or more fluids, a distal portion 1314, and an overlapping portion positioned along the first porous element 1310 between the proximal portion 1312 and the distal portion 1314. The second porous element 1320 includes a proximal portion 1322 configured to receive one or more fluids, a distal portion 1324, and an overlapping portion positioned along the second porous element 1320 between the proximal portion 1322 and the distal portion 1324.

The proximal portion 1312 of the first porous element 1310 can include an input portion 1316. For example, in the embodiment shown in FIG. 12A, the input portion 1316 comprises a separate porous element, such as a source pad (e.g., glass fiber, nitrocellulose, etc.) that is positioned in fluid communication with the first porous element 1310 and configured to receive a volume of fluid from a fluid source (e.g., a transfer pipette). In other embodiments, the proximal portion 1312 does not include a separate input portion 1316, and the fluid can be delivered directly to the proximal portion 1312 of the first porous element 1310 and/or one or more fluid sources (such as a well) can be positioned on the first porous element 1310.

The distal portion 1314 of the first porous element 1310 can include a receiving portion 1318. For example, in the embodiment shown in FIG. 12A, the receiving portion 1318 comprises a waste pad (e.g., cellulose, nitrocellulose, etc.) positioned in fluid communication with the first porous element 1310 and configured to receive all or a portion of the delivered volume of the first fluid F₁ after it travels through the first porous element 1310. In other embodiments, the distal portion 1314 does not include a separate receiving portion 1318.

The proximal portion 1322 of the second porous element 1320 can include an input portion 1326. For example, in the embodiment shown in FIG. 12A, the input portion 1326 comprises a well that is positioned in fluid communication with the second porous element 1320 and configured to receive a volume of fluid from a fluid source (e.g., a transfer pipette). In other embodiments, the input portion 1326 comprises a separate porous element such as a source pad (e.g., glass fiber, nitrocellulose, cellulose, etc.) that is positioned in fluidic communication with the second porous element 1320. In yet other embodiments, the proximal portion 1322 does not include a separate input portion 1326, and the fluid can be delivered directly to the proximal portion 1322 of the second porous element 1320.

The distal portion 1324 of the second porous element 1320 can include a receiving portion 1328. For example, in the embodiment shown in FIG. 12A, the receiving portion 1328 comprises a waste pad (e.g., cellulose, nitrocellulose, etc.) positioned in fluid communication with the second porous element 1320 and configured to receive all or a portion of the volume of second fluid F₂ after it travels through the second porous element 1320. In other embodiments, the distal portion 1324 does not include a separate receiving portion 1328.

The overlapping portion of the first porous element 1310 and the overlapping portion of the second porous element 1320 together define the overlapping region 1302. For example, the cross-sectional area of the overlapping region 1302 is generally equal to the width of the first porous element 1310 multiplied by the width of the second porous element 1320.

FIG. 12B is a cross-sectional end view of a portion of the device 1300 taken along the line 12B-12B in FIG. 12A. Referring to FIGS. 12A-12B together, the second porous element 1320 includes a proximal top member 1320a and a distal bottom member 1320b. Within the overlapping region 1302, the first porous element 1310 can be positioned between a distal region of the proximal top member 1320a and a proximal region of the distal bottom member 1320b. As such, the top and bottom members 1320a, 1320b of the second porous element 1320 can be in direct contact with the first porous element 1310 within the overlapping region 1302. Accordingly, the overlapping portion of the first porous element 1310 is in fluid communication with the proximal top member 1320a and the distal bottom member 1320b of the second porous element 1320. In some embodiments the first and second porous elements 1310, 1320 can be spaced apart (e.g., by one or more materials), so long as the overlapping portion of the first porous element 1310 remains in fluid communication with the overlapping portion of the second porous element 1320.

The first porous element 1310 and the second porous element 1320 can be selected based on their respective WRC's for a given fluid and medium, As shown in the graph of FIG. 9C, for the same fluid and medium, the second porous element 1320 has a WRC that is shifted to the left of the first porous element 1310. In other words, for the same fluid, medium, and volumetric water content, the second porous element 1320 exhibits a lower suction pressure than the first porous element 1310. Each of the porous element's WRC represents a novel, advanced quantitative model that compiles several parameters affecting the negative pressure magnitude at the overlapping region 1302, such as contact angle between the delivered fluid and the respective porous element and average pore size of the respective porous element. In the embodiment shown in FIGS. 12A-12B. The first porous element 1310 is made of a first porous material having a first average pore size, and the second porous element 1320 is made of a second porous material having a second average pore size greater than the first average pore size. In the embodiment shown in FIGS. 12A-12B, for example, the first porous element 1310 is made of nitrocellulose, and the second porous element 1320 is made of glass fiber. In other embodiments, the first and second porous elements 1310, 1320 can be made of other porous materials so long as the WRC of the second porous element 1320 is shifted to the left of the WRC of the first porous element 1310.

As shown in FIGS. 12A-12B, the overlapping portion of the first porous element 1310 can be impregnated with one or more capture molecules 1306 configured to selectively adhere a targeted analyte or target molecule of a biological sample within the first and/or second fluid as the first and/or second fluid moves through the overlapping region. For example, in DNA-recovering applications of the present technology, the first porous element 1310 can be impregnated with a DNA capture molecule (such as chitosan), and the first fluid F₁ can be a 5-10 mL urine sample containing the targeted DNA. As shown in the time-lapsed views of the microfluidic device 1300 shown in FIGS. 13A-13F, after delivery of all or a portion of the biological sample F₁ to the proximal portion 1312 of the first porous element 1310, the biological sample F₁ wicks distally through the first porous element 1310, towards the overlapping region 1302. Because of the fluid pressure at the overlapping region 1302, the wicking front WF of the first fluid F₁ passes through the overlapping region 1302 without wetting the overlapping portion of the second porous element 1320, as shown in FIGS. 13C-13D. As the first fluid F₁ passes over and/or through the capture molecules (not visible in FIGS. 13A-13E), analyte or target molecule in the sample F₁ remains trapped within the overlapping region 1302 while the remaining contaminants and fluid flow to waste. In some embodiments, a wash fluid can also be delivered to the first porous element 1310.

Once the distal portion of the first porous element 1310 (e.g., waste pad 1318) becomes sufficiently saturated, a second fluid F₂ configured to release the analyte or target molecule from the capture molecules 1306 can be delivered to the input portion 1326 of the second porous element 1320. For example, in the DNA-recovery example provided above, the second fluid F₂ can be an elution buffer. Addition of the second fluid F₂ to the second porous element 1320 lowers the magnitude of the pressure at the overlapping region 1302, thereby allowing the second fluid F₂ to imbibe distally along the second porous element 1320. As shown in FIGS. 13E-13F, as the second fluid F₂ passes through the overlapping region 1302, the second fluid F₂ mixes with first fluid F₁ and pulls the first fluid F₁ distally along the second porous element 1320 to the clean distal portion 1324, from which the captured and/or purified analyte or targeted molecule can be quantified (e.g., captured and/or purified DNA can be quantified via qPCR).

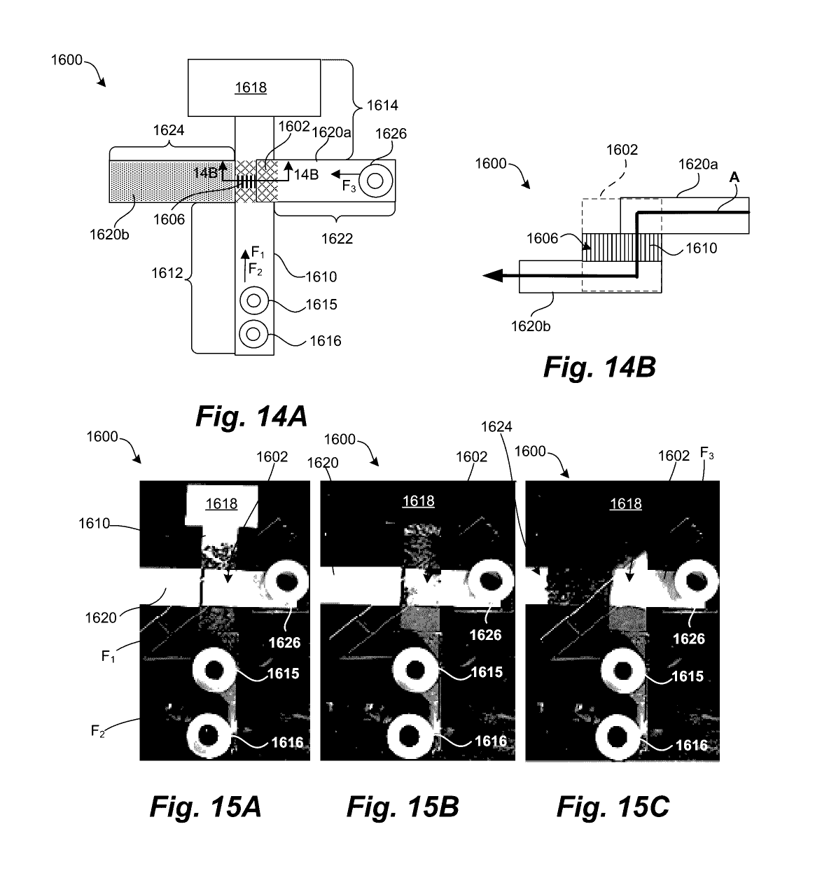

FIGS. 14A-14B are top and cross-sectional end views, respectively, of a paper-based microfluidic device 1600 (also referred to herein as “device 1600”) configured in accordance with yet another embodiment of the present technology. The microfluidic device 1600 of FIGS. 14A-14B can be generally similar to the microfluidic device 1300 shown in FIGS. 12A-12B, except the microfluidic device 1600 includes a first fluid source 1615 and a second fluid source 1616 positioned at and spaced apart along the proximal portion 1612 of the first porous element 1610. Each of the first and second fluid sources 1615, 1616 can have an outlet in fluid communication with the proximal portion of the first porous element 1610. The first and second fluid sources 1615, 1616 are configured to sequentially deliver a first and second fluid to the first porous element 1610. Additional details regarding sequential delivery of fluid volumes is described in International Patent Application No. PCT/US14/12618, filed Jan. 22, 2014, which is incorporated herein by reference in its entirety.

As shown in the time-lapsed views of the microfluidic device 1600 shown in FIGS. 15A-15C, first fluid source 1615 containing the first fluid F₁, or sample containing the analyte or target molecule of interest, and the second fluid source 1616 containing the second fluid F₂, or wash buffer, are simultaneously placed in fluid communication with the first porous element 1610. The sample F₁ and wash buffer F₂ wick sequentially through the first porous element 1610 towards the overlapping region 1602. Because the second porous element 1620 has a greater average pore size than the first porous element 1610, the fluid pressure within the overlapping region 1602 exceeds the capillary pressure P_{c} of the second porous element 1620. As such, the wicking fronts of the sample F₁ and the wash buffer F₂ pass through the overlapping region 1602 without wetting the overlapping portion of the second porous element 1620, as shown in FIG. 15A. As the sample F₁ passes over and/or through the capture molecules (not visible in FIGS. 15A-15C), the analyte or target molecule of interest in the sample F₁ remains trapped within the overlapping region 1602 while the remaining contaminants and fluid flow to waste.

Once the distal portion 1614 and/or receiving region 1618 of the first porous element 1610 becomes sufficiently saturated, a third fluid F₃, such as an elution buffer, can be delivered to the input portion 1626 of the second porous element 1620. Addition of the third fluid F₃ to the second porous element 1620 lowers the pressure at the overlapping region 1602, thereby allowing the third fluid F₃ to imbibe distally along the second porous element 1620. As shown in FIG. 15C, as the third fluid F₃ passes through the overlapping region 1602, the third fluid F₃ mixes with the second fluid F₂ and pulls the second fluid F₂ distally along the second porous element 1620 to the clean distal portion 1624, from which the captured and/or purified analyte or targeted molecule can be quantified (e.g., captured and/or purified DNA can be quantified via qPCR).

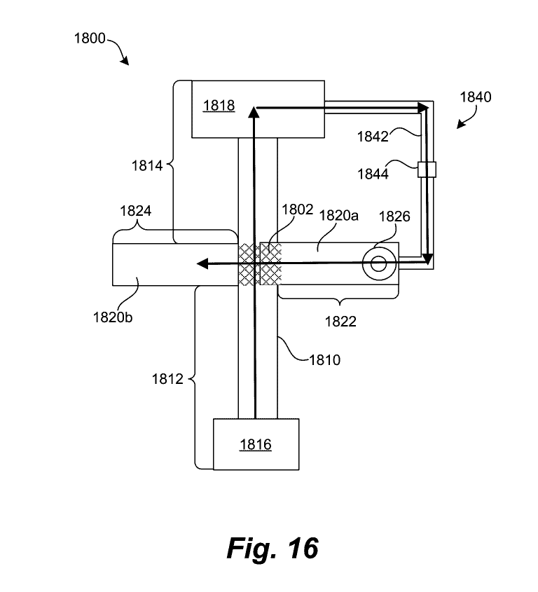

FIG. 16 is a top view of another embodiment of a paper-based microfluidic device 1800 (also referred to herein as “device 1800”) configured in accordance with the present technology. The microfluidic device 1800 of FIG. 16 can be generally similar to the microfluidic device 1300 shown in FIGS. 12A-12B, except the microfluidic device 1800 includes a fluid-activated actuator 1840 for automating delivery of the second fluid to the second porous element 1820. The actuator 1840 can include a mechanical linkage 1842 (shown schematically in FIG. 16) and a fluid-activated switch 1844. The fluid-activated switch can be in fluid communication with the waste pad 1818 and/or distal portion 1814 of the first porous element 1810 such that a predetermined saturation level of the waste pad 1818 and/or distal portion 1814 triggers the release of the second fluid to the second porous element 1820.

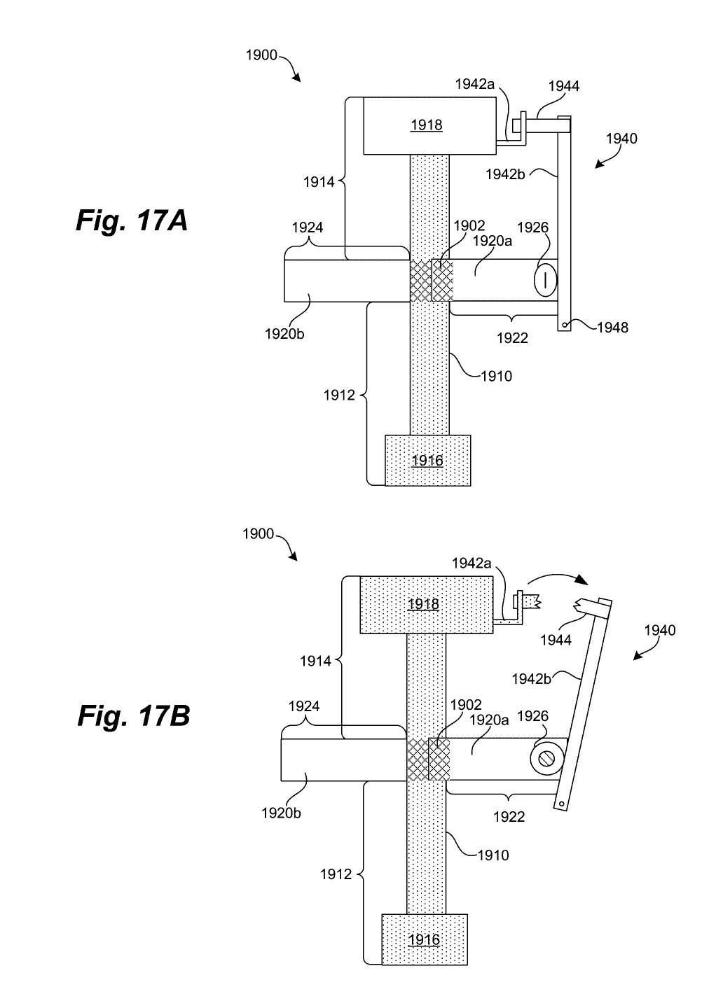

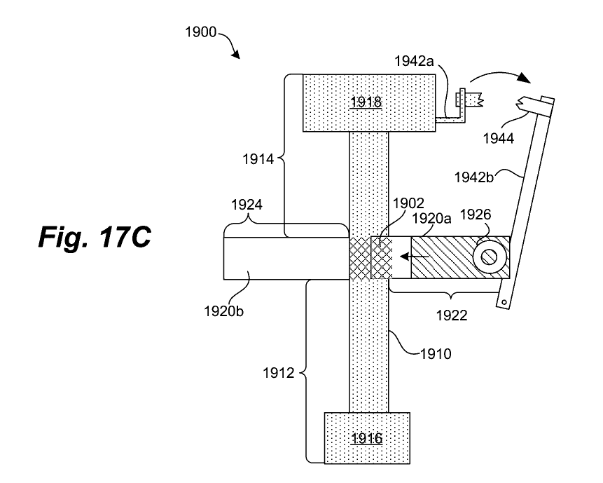

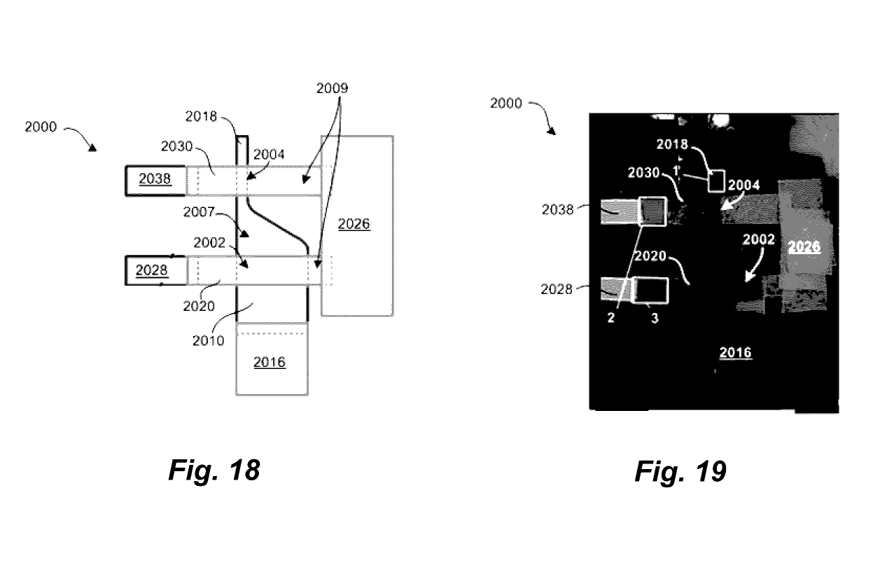

FIG. 17A-17C are time-lapsed views of one example of a microfluidic device 1900 including a fluidic actuator 1940 configured in accordance with the present technology. The microfluidic device 1900 can be generally similar to the microfluidic device 1800 of FIG. 18. As shown in FIGS. 17A-17C, the actuator 1940 includes a linkage 1942a, an arm 1942b, and a dissolvable pathway 1944 therebetween. The linkage 1942a can be in fluid communication with the distal portion 1914 and/or waste pad 1918 of the first porous element 1910. The arm 1942b is rotatable around a hinge point 1948 between a first, strained position (shown in FIG. 17A) and a second, relaxed position (shown in FIG. 17B). In some embodiments, the arm 1942b can be spring-biased towards its resting position. In the first, strained position, the arm 1942b is held in place by the dissolvable pathway 1944, and a portion of the arm 1942b pinches the second fluid source 1926 closed such that the second fluid is prevented from contacting the second porous element 1920. As shown in FIGS. 17B-17C, once the receiving region 1918 and/or the distal portion 1914 of the first porous element 1910 becomes sufficiently saturated the dissolvable pathway will break apart, thereby releasing the arm 1942b and placing the second fluid source 1926 in fluid communication with the second porous element 1920.

### IV. Selected Embodiments of Pressure-Based Microfluidic Devices for Automating Dilution

The pressure-based microfluidic devices and systems disclosed herein can include intersecting networks of porous elements. For example, fluidic systems of the present technology may include a first porous network comprising one or more first porous elements, and a second porous network comprising one or more second porous elements that intersect of overlap one or more of the first porous elements. Such a configuration, for example, may be utilized for delivering differing quantities of analyte to assay zones arranged in parallel. FIG. 18, for example, shows one embodiment of a microfluidic device 2000 configured for serial dilutions. As shown in FIG. 18, the device 2000 can include a first porous network 2007 supplied by a first fluid source 2016 and a second porous network 2009 supplied by a second fluid source 2026. The first porous network 2007 can include a single porous element 2010 (not including source pads or waste pads) or single stack of porous elements, and the second porous network 2009 can include a first porous element 2020 and a second porous element 2030 spaced apart from the first porous element 2020. The first and second porous elements 2020, 2030 can be positioned across the porous element 2010 to create overlapping regions 2002, 2004.

The porous element(s) of the second network 2009 can have a WRC that is shifted to the left of the WRC of the porous element(s) of the first network 2007. For example, in some embodiments, the porous element 2010 can be nitrocellulose and each of the first and second porous elements 2020, 2030 can be glass fiber.

It will be appreciated that although the embodiment of FIG. 18 shows the first network 2007 having only a single porous element, in other embodiments the first network 2007 can include more than one porous element (e.g., two porous elements, three porous elements, etc.). Likewise, although the embodiment of FIG. 18 shows the second network 2009 having two porous elements, in other embodiments the second network 2009 can include more or fewer porous elements (e.g., one porous element, three porous elements, etc.).

FIG. 19 shows the device 2000 of FIG. 18 after a first fluid has been delivered to the first network 2007 (e.g., via source pad 2016) and a second fluid has been delivered to the second network 2009 (e.g., via source pad 2026), in a method generally similar to that described above with reference to FIGS. 10A-10D. As shown in FIG. 19, the arrangement of porous elements of FIG. 18 produces three different concentrations of the first fluid (identified by boxed, numbered regions of the device 2000): 1) non-diluted, 2) somewhat diluted, 3) more diluted.

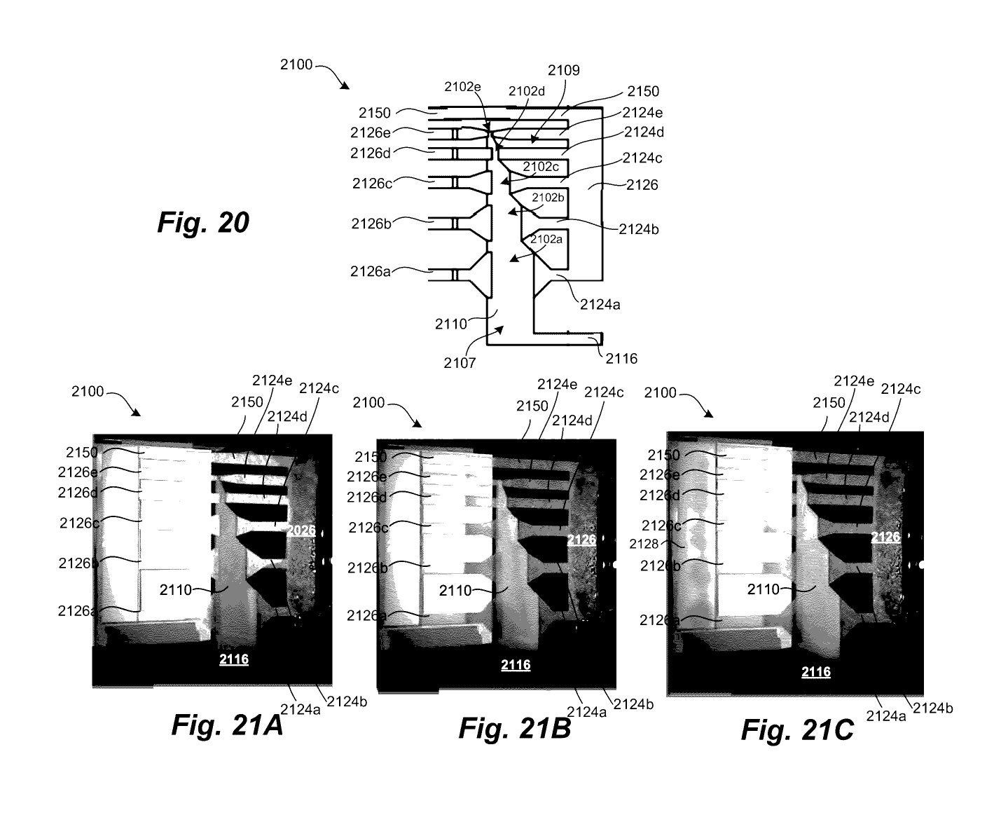

FIG. 20 illustrates another embodiment of a microfluidic device 2100 having multiple overlapping regions for serial dilutions configured in accordance with the present technology. The device 2100 can include a first porous network 2107 supplied by a first fluid source 2116 and a second porous network 2109 supplied by a second fluid source 2126. The first porous network 2107 can include a single porous element 2110 (not including source pads or waste pads) or single stack of porous elements, and the second porous network 2109 can include five second porous elements, each having a proximal leg 2124a-2124e and a distal leg 2126a-2126e. A negative control 2150 can optionally be included which delivers no sample. The second porous elements can be positioned on either side of the porous element 2010 to create overlapping regions 2102a-2102e. As shown in FIG. 20, one or more of the overlapping regions 2102a-2102e and/or distal and proximal legs 2124a-2124e, 2126a-2126e can have the same or different shapes and/or sizes.

FIGS. 21A-21C are time-lapsed views of the microfluidic device 2100 after a first fluid has been delivered to the first network 2107 (e.g., via source pad 2116) and a second fluid has been delivered to the second network 2109 (e.g., via source pad 2126), in a method generally similar to that described above with reference to FIGS. 10A-10D. As best illustrated by FIG. 21 C, the arrangement of porous elements of FIG. 20 produces five different concentrations of the first fluid (as demonstrated by the waste pad 2128 at FIG. 21C).

### V. Conclusion

In some embodiments, any of the pathways disclosed herein can include additional first and/or second materials in series along the same pathway connected by an additional flow-metering element (not shown). Further, in particular embodiments, a single pathway can have multiple branches (not shown) that converge and/or diverge. Examples of these and other suitable pathways and/or capillarity devices are described in International Patent Application No. PCT/US2010/061675, filed Dec. 21, 2010, and International Patent Application No. PCT/US2012/044060, filed Jun. 6, 2012, both of which are incorporated herein by reference in their entireties.

From the foregoing it will be appreciated that, although specific embodiments of the technology have been described herein for purposes of illustration, various modifications may be made without deviating from the spirit and scope of the technology. For example, the presence/configuration of the base or housing, the number of pathways, flow-metering elements, volume-metering features, the use of pre-wetted pads, the specific types of fluids, and the material choices for various components of the devices described above with reference to FIGS. 1A-21C may vary in different embodiments of the technology. Further, certain aspects of the new technology described in the context of particular embodiments may be combined or eliminated in other embodiments. For example, in the embodiments illustrated above, various combinations of flow-metering and volume-metering elements or features may be combined into a single device. Moreover, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein. Thus, the disclosure is not limited except as by the appended claims.

I/We claim:

1. A method for controlling fluid flow through a fluidic network including a first porous element and a second porous element, wherein each of the first and second porous elements have a proximal portion and a distal portion, the method comprising:
delivering a first fluid to the proximal portion of the first porous element, wherein the first porous element is positioned across the second porous element such that an overlapping region exists between the first and second porous elements, and wherein
the overlapping region includes an overlapping portion of the first porous element and an overlapping portion of the second porous element,
the overlapping region is between (a) the proximal and distal portions of the first porous element, and (b) the proximal and distal portions of the second porous element, and
the first and second porous elements are in fluid communication with one another at the overlapping region; and
pulling the first fluid distally along the first porous element and through its overlapping portion, wherein the first fluid moves through the overlapping region without wetting the overlapping portion of the second porous element;
after at least a portion of the first fluid has moved through the overlapping region, delivering a second fluid to the proximal portion of the second porous element;
mixing the first and second fluids at the overlapping region; and
pulling the first and second fluids into the distal portion of the second porous element.

2. The method of claim 1 wherein delivering the second fluid to the proximal portion of the second porous element changes a fluid pressure differential at the overlapping region.

3. The method of claim 1 wherein the first porous element has a first average pore size and the second porous element has a second average pore size greater than the first average pore size.

4. The method of claim 1 wherein delivering the first fluid to the proximal portion of the first porous element includes delivering between about 0.1 mL and about 50 mL of the first fluid to the proximal portion of the first porous element.

5. The method of claim 1 wherein delivering the first fluid to the proximal portion of the first porous element includes delivering between about 1 mL and about 15 mL of the first fluid to the proximal portion of the first porous element.

6. The method of claim 1 wherein delivering the first fluid to the proximal portion of the first porous element includes delivering between about 5 mL and about 10 mL of the first fluid to the proximal portion of the first porous element.

7. The method of claim 1 wherein at least a portion of the overlapping region is impregnated with a capture molecule configured to selectively adhere a component of a biological sample within the first and/or second fluid as the first and/or second fluid moves through the overlapping region.

8. The method of claim 1, further comprising fluidly connecting the distal portion of the first porous element to the proximal portion of the second porous element.

9. The method of claim 1, further comprising automatically fluidly connecting the distal portion of the first porous element to the proximal portion of the second porous element when the distal portion of the first porous element reaches a predetermined level of saturation of the first fluid.

10. A microfluidic device for performing one or more chemical processes, the device comprising:
a first porous element having a proximal portion, and a distal portion opposite the proximal portion, wherein the first element has a first average pore size and is configured to receive a first fluid at its proximal portion; and
a second porous element having a proximal portion, a distal portion opposite the proximal portion, and a second average pore size greater than the first average pore size, wherein the second porous element is configured to receive a second fluid at its proximal portion,
wherein the first porous element is positioned across the second porous element such that an overlapping region exists between the first and second porous elements, and wherein
an overlapping portion of the first porous element is between the proximal and distal portions of the first porous element,
an overlapping portion of the second porous element is between the proximal and distal portions of the second porous element,
the overlapping portion of the first porous element is in fluid communication with the overlapping portion of the second porous element, and
wherein, before the second fluid is delivered to the proximal portion of the second porous element, delivery of the first fluid to the proximal portion of the first porous element causes the first fluid to travel distally through the overlapping portion of the first porous element without wetting the overlapping portion of the second porous element.

11. The microfluidic device of claim 10, further comprising a capture molecule positioned within the overlapping region, wherein the capture molecule is configured to selectively adhere a component of a biological sample within the first and/or second fluid.

12. The microfluidic device of claim 11 wherein the capture molecule is a linear polysaccharide configured to bind one or more nucleic acids in a pH-dependent manner.

13. The microfluidic device of claim 11 wherein the capture molecule is chitin or chitosan.

14. The microfluidic device of claim 10 wherein the second porous element is impregnated with nucleic acid amplification reagents.

15. The microfluidic device of claim 10, further comprising a first wicking pad at the distal portion of the first porous element and a second wicking pad at the distal portion of the second porous element.

16. The microfluidic device of claim 10 wherein the first porous element is configured to receive at least 1 mL of the first fluid.

17. The microfluidic device of claim 10 wherein the first porous element is configured to receive from about 0.1 mL to about 50 mL of the first fluid.

18. The microfluidic device of claim 10 wherein the first porous element is configured to receive from about 1 mL to about 10 mL of the first fluid.

19. The microfluidic device of claim 10 wherein the first porous element is configured to receive from about 5 mL to about 10 mL of the first fluid.

20. The microfluidic device of claim 10 wherein at least one of the first fluid source and the second fluid source is a well having negligible capillary backpressure, and wherein the well has an outlet that is in fluid communication with the corresponding first or second porous element.

21. The microfluidic device of claim 10 wherein the second porous element includes a first porous member positioned below the first porous element and a second porous member positioned above the first porous element, and wherein at least a portion of each of the first and second porous members is positioned within the overlapping region.

22. The microfluidic device of claim 10, further comprising a valve positioned between the distal portion of the first porous element and the proximal portion of the second porous element.

23. The microfluidic device of claim 22 wherein the valve is configured to automatically fluidly connect the distal portion of the first porous element and the proximal portion of the second porous element in response to a predetermined saturation level of the distal portion of the first porous element by the first fluid.

24. The microfluidic device of claim 10, further comprising:
a barrier between the distal portion of the first porous element and the proximal portion of the second porous element; and
a puncturing element configured to puncture the barrier to fluidly connect the distal portion of the first porous element and the proximal portion of the second porous element.
